(19) United States
(12) Patent Application Publication
(10) Pub. No.: US 2024/0130742 A1
(43) Pub. Date: Apr. 25, 2024

(54) IMPLANTS FOR BONE FIXATION OR FUSION

(71) Applicant: SI-Bone Inc., Santa Clara, CA (US)

(72) Inventors: Mark A. REILEY, Delray Beach, FL (US); Bret SCHNEIDER, San Jose, CA (US); Joanne LEUNG, Mountain View, CA (US); Paul M. SAND, Redwood City, CA (US); Scott A. YERBY, Montara, CA (US)

(21) Appl. No.: 18/317,832

(22) Filed: May 14, 2023

Related U.S. Application Data
(63) Continuation of application No. 16/550,032, filed on Aug. 23, 2019, now Pat. No. 11,684,378.
Application No. 16/550,032 is a continuation of application No. 16/237,409, filed on Dec. 31, 2018, now abandoned.
Application No. 16/237,409 is a division of application No. 14/859,046, filed on Sep. 18, 2015, now Pat. No. 10,166,033.
(60) Provisional application No. 62/052,318, filed on Sep. 18, 2014.

(51) Int. Cl. A61B 17/17; A61B 17/16; A61B 17/84; A61F 2/44

(52) U.S. Cl. CPC A61B 17/1757; A61B 17/1604; A61B 17/84; A61F 2/44; A61F 2002/30995

(57) ABSTRACT

Implants for the fusion or fixation of two bone segments are described. For example, the implants can be used for the fusion or fixation of the sacroiliac joint. The implants can include fenestrations, have a rectilinear overall cross-sectional area, and have a curvature. Some implants can also be used to rescue failed implants.

### CROSS REFERENCE TO RELATED APPLICATIONS

This application is a continuation of U.S. application Ser. No. 16/550,032, filed Aug. 23, 2019, titled “IMPLANTS FOR BONE FIXATION OR FUSION”, which is a continuation of U.S. application Ser. No. 16/237,409 filed Dec. 31, 2018, titled “IMPLANTS FOR BONE FIXATION OR FUSION”, now abandoned, which is a divisional of U.S. application Ser. No. 14/859,046 filed Sep. 18, 2015, titled “IMPLANTS FOR BONE FIXATION OR FUSION”, now U.S. Pat. No. 10,166,033 which claims priority to U.S. Provisional Patent Application No. 62/052,318, filed Sep. 18, 2014, titled “IMPLANTS FOR BONE FIXATION OR FUSION”, each of which are herein incorporated by reference in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the invention relate generally to bone implants that can be used to fuse two bone segments together.

### BACKGROUND

Many types of hardware are available both for the fixation of bones that are fractured and for the fixation of bones that are to be fused (arthrodesed).

For example, the human hip girdle (see FIGS. 1 and 2) is made up of three large bones joined by three relatively immobile joints. One of the bones is called the sacrum and it lies at the bottom of the lumbar spine, where it connects with the L5 vertebra. The other two bones are commonly called “hip bones” and are technically referred to as the right ilium and the left ilium. The sacrum connects with both hip bones at the sacroiliac joint (in shorthand, the SI-Joint).

The SI-Joint functions in the transmission of forces from the spine to the lower extremities, and vice-versa. The SI-Joint has been described as a pain generator for up to 22% of lower back pain patients.

To relieve pain generated from the SI-Joint, sacroiliac joint fusion is typically indicated as surgical treatment, e.g., for degenerative sacroiliitis, inflammatory sacroiliitis, iatrogenic instability of the sacroiliac joint, osteitis condensans ilii, or traumatic fracture dislocation of the pelvis. Currently, screws and screws with plates are used for sacro-iliac fusion. At the same time the cartilage has to be removed from the “synovial joint” portion of the SI-Joint. This requires a large incision to approach the damaged, subluxed, dislocated, fractured, or degenerative joint. The large incision and removal of tissue can cause significant trauma to the patient, resulting in pain and increasing the time to heal after surgery.

In addition, screw type implants tend to be susceptible to rotation and loosening, especially in joints that are subjected to torsional forces, such as the SI-Joint. Excessive movement of the implant after implantation may result in the failure of the implant to incorporate and fuse with the bone, which may result in the need to remove and replace the failed implant.

Consequently, it would be desirable to provide an implant for bone fusion or fixation that resists rotation, can be implanted using a minimally invasive procedure, and/or that can be used to rescue a failed implant.

### SUMMARY OF THE DISCLOSURE

The present invention relates generally to bone implants that can be used to fuse two bone segments together.

In some embodiments, an implant for the fixation or fusion of the SI-Joint is provided. The implant can include an elongate body having a longitudinal axis and a noncircular cross-sectional profile transverse to the longitudinal axis, the elongate body having a proximal end and a distal end, wherein the elongate body is curved along the longitudinal axis from the proximal end to the distal end of the elongate body.

In some embodiments, the noncircular cross-sectional profile has one or more apices. In some embodiments, the noncircular cross-sectional profile is substantially rectilinear. In some embodiments, the noncircular cross-sectional profile is triangular.

In some embodiments, the elongate body has a curvature between about 5 and 45 degrees. In some embodiments, the elongate body has a curvature between about 15 and 30 degrees.

In some embodiments, the elongate body has a plurality of fenestrations. In some embodiments, the fenestrations are located on a distal portion of the elongate body.

In some embodiments, a method of implanting a curved implant across a joint or fracture between a first bone segment and a second bone segment is provided. The method can include forming a curved insertion path from the first bone segment, across the joint or fracture between the first bone segment and the second bone segment, and to the second bone segment, wherein the curved insertion path has a predetermined radius of curvature along its length; and inserting a curved implant having a matching radius of curvature into the curved insertion path and into the first bone segment, across the joint or fracture, and into the second bone segment.

In some embodiments, the method further includes inserting a curved guidewire alone the curved insertion path. In some embodiments, the curved guidewire is rotated into the curved insertion path.

In some embodiments, the method further includes disposing a drill bit over the curved guidewire; and drilling a curved bore along the curved guidewire.

In some embodiments, the method further includes disposing a broach over the curved guidewire; and shaping the curved bore with the broach to form the insertion path.

In some embodiments, both the shaped insertion path and the curved implant have a transverse cross-sectional profile that is rectilinear. In some embodiments, both the shaped insertion path and the curved implant have a transverse cross-sectional profile that is defined by at least one apex. In some embodiments, both the shaped insertion path and the curved implant have a transverse cross-sectional profile that is triangular. In some embodiments, both the shaped insertion path and the curved implant have a transverse cross-sectional profile that is rectangular.

In some embodiments, the method further includes disposing a sharp tipped broach over the curved guidewire; and creating a curved and shaped bore along the curved guidewire using the sharp tipped broach.

In some embodiments, an implant for the fixation or fusion of the SI-Joint is provided. The implant can include an elongate body have a longitudinal axis and a noncircular cross-sectional profile transverse to the longitudinal axis, the elongate body having a proximal end and a distal end, wherein the elongate body comprises one or more faces that extend from the proximal end to the distal end of the elongate body, each face having a slot extending from the distal end of the elongate body towards the proximal end of the elongate body along the longitudinal axis; and a rib slidably disposed in each slot.

In some embodiments, the slot is selected from the group consisting of a dovetail slot and a t-slot.

In some embodiments, the rib is made of a material selected from the group consisting of bone graft, metal, metal alloy, hydroxyapatite, ceramic, and polymer.

In some embodiments, the elongate body has three faces and a triangular cross-sectional profile transverse to the longitudinal axis.

In some embodiments, the rib is wedge shaped.

In some embodiments, the rib is positioned in a central portion of the implant and offset from both the proximal end and the distal end of the elongate body.

In some embodiments, an implant for the fixation or fusion of the SI-Joint is provided. The implant can include an elongate body have a longitudinal axis and a noncircular cross-sectional profile transverse to the longitudinal axis having one or more apices, wherein a portion of the one or more apices is removed to form a face along the one or more apices, the face having a slot that extends along the longitudinal axis; and a rib slidably disposed in the slot.

In some embodiments, the rib is made of a material selected from the group consisting of bone graft, metal, metal alloy, hydroxyapatite, ceramic, and polymer.

In some embodiments, the rib is sized and configured to restore the shape of the removed portion of the one or more apices.

In some embodiments, an implant for the fixation or fusion of the SI-Joint is provided. The implant can include an elongate body have a longitudinal axis and a noncircular cross-sectional profile transverse to the longitudinal axis, the elongate body having a proximal end and a distal end, wherein the elongate body comprises one or more faces that extend from the proximal end to the distal end of the elongate body; an elongate rib disposed along the one or more faces of the elongate body; and a wire cage configured to secure the elongate rib to the elongate body.

In some embodiments, the wire cage comprises a first opening to receive the elongate body and a second opening to receive the elongate rib.

In some embodiments, an implant for the fixation or fusion of the SI-Joint is provided. The implant can include an elongate body have a longitudinal axis and a noncircular cross-sectional profile transverse to the longitudinal axis, the elongate body having a proximal end and a distal end, wherein the elongate body comprises one or more faces that extend from the proximal end to the distal end of the elongate body; and one or more fenestrations located on a distal portion of the elongate body.

In some embodiments, the one or more fenestrations are located along a central portion of the one or more faces of the elongate body.

In some embodiments, the elongate body comprises at least one apex and the one or more fenestrations are located at the distal end of the at least one apex.

In some embodiments, the one or more fenestrations are circular.

In some embodiments, the one or more fenestrations are oblong.

In some embodiments, the elongate body is tapered such that the distal end has a smaller diameter or width than the proximal end.

In some embodiments, the elongate body comprises a plurality of walls that form the one or more faces and define a lumen having a noncircular cross-sectional profile.

In some embodiments, the noncircular cross-sectional profile of both the elongate body and the lumen is triangular.

In some embodiments, the noncircular cross-sectional profile of both the elongate body and the lumen is rectangular.

In some embodiments, the elongate body is coated with hydroxyapatite.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1 and 2 are, respectively, anterior and posterior anatomic views of the human hip girdle comprising the sacrum and the hip bones (the right ilium, and the left ilium), the sacrum being connected with both hip bones at the sacroiliac joint (in shorthand, the SI-Joint).

FIGS. 3 and 4 are embodiments of various straight implants that can be used for the fusion or fixation of a joint or two bone segments.

FIG. 5 illustrates an axial section view of the SI-Joint with an implant for the fixation of the SI-Joint using a lateral approach that goes laterally through the ilium, the SI-Joint, and into the sacrum.

FIG. 6 illustrates an axial section view of the SI-Joint with an implant for the fixation of the SI-Joint using a postero-lateral approach entering from the posterior iliac spine of the ilium, angling through the SI-Joint, and terminating in the sacral alae.

FIGS. 7A-7M illustrate perspective views of alternative embodiments of implants that can be used for the fusion or fixation of a joint or two bone segments.

FIG. 8 is a cross-sectional view of an embodiment of an implant having a triangular thru cannula.

FIGS. 9A-9B illustrate an embodiment of an implant with larger dimensions and truncated corners.

FIGS. 10A-10B illustrate an embodiment of an implant with a plurality of ribs.

FIG. 10C illustrates another embodiment of an implant with a plurality of ribs that are formed by osteoconductive materials such as bone, metal, metal alloy, ceramic, and polymer.

FIGS. 10D-10F illustrate additional embodiments of various implants with ribs.

FIGS. 11A-11C illustrate additional embodiments of implants with ribs.

FIGS. 12A and 12B illustrate an embodiment of an implant with a double triangle cross-sectional profile.

FIGS. 13A and 13B illustrate another embodiment of an implant with ribs.

FIGS. 14A and 14B illustrate another embodiment of a triangular implant with curved sides.

FIGS. 15A and 15B illustrate an embodiments of an implant that has been coated with a titanium plasma spray and a hydroxyapatite coating.

FIG. 16 illustrates the use of curved tooling for the implantation of a curved implant.

FIGS. 17A-17C illustrate various views of a modified broach for creating a bore in bone suitable for receiving an implant with ribs.

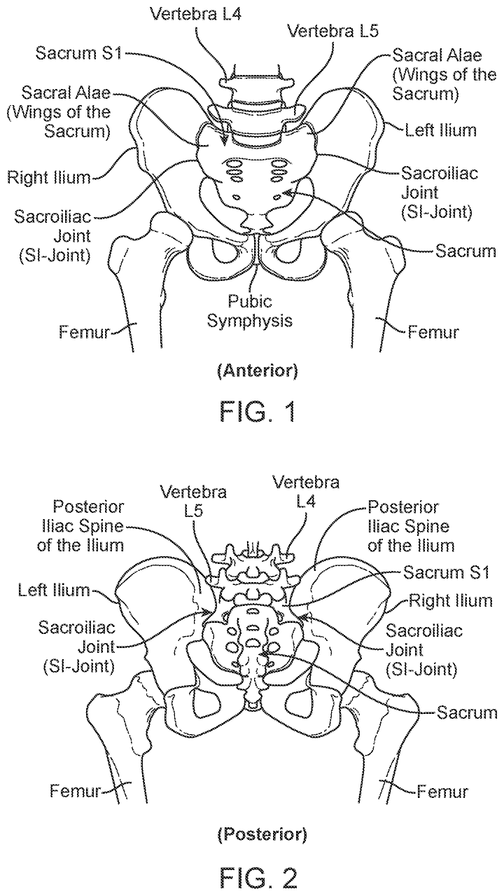

### DETAILED DESCRIPTION

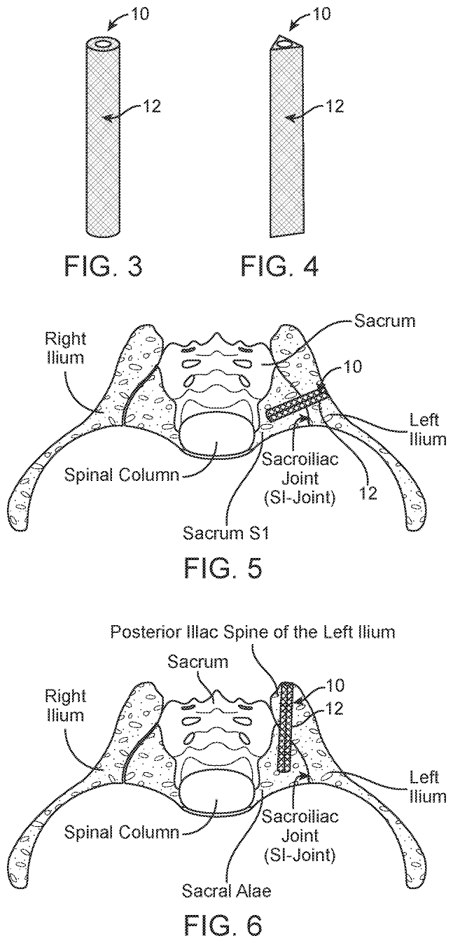

FIG. 3 and FIG. 4 illustrate straight implants 10 with a solid elongate body 12 that can be used for the fixation or fusion of two bone segments. The implant 10 shown in FIG. 3 is cylindrical and can optionally have screw threads along the exterior of the implant body. As mentioned above, cylindrical screw type implants can suffer from excessive rotation. One solution to this problem is the implant 10 in FIG. 4, which has a non-cylindrical cross-sectional area. For example, as shown, the implant 10 can have a triangular cross-sectional area, although other rectilinear cross-sectional profiles may be used as well, including rectangular, hexagonal and the like. Non-cylindrical implants need not have a strict rectilinear cross-sectional profile in order to resist rotation. A cross-sectional area that is non-circular will generally suffice. For example, a tear drop shaped cross-sectional area, or a cross-sectional area with at least one apex, can resist rotation. Other non-circular cross-sectional geometries that may not have a rectilinear component can also work, such as oval cross-sections.

FIG. 5 illustrates insertion of the implant 10 of FIG. 4 across the SI-Joint using a lateral approach that goes laterally through the ilium, across the SI-Joint, and into the sacrum. FIG. 6 illustrates insertion of the same implant across the SI-Joint using a postero-lateral approach entering from the posterior iliac spine of the ilium, angling through the SI-Joint, and terminating in the sacral alae. Many of the implants described herein can be inserted across the SI-Joint in a similar manner.

### Implant Variations

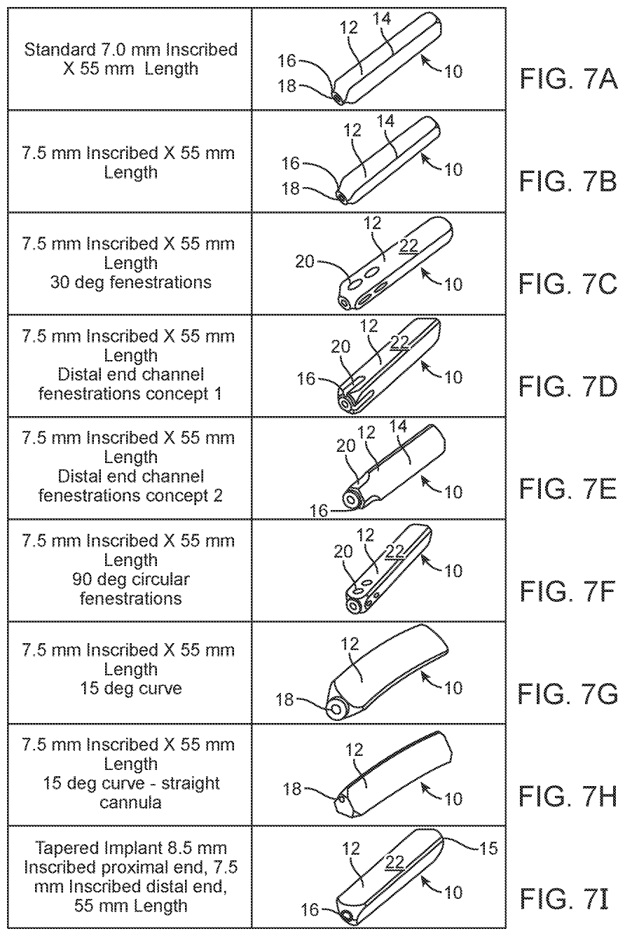

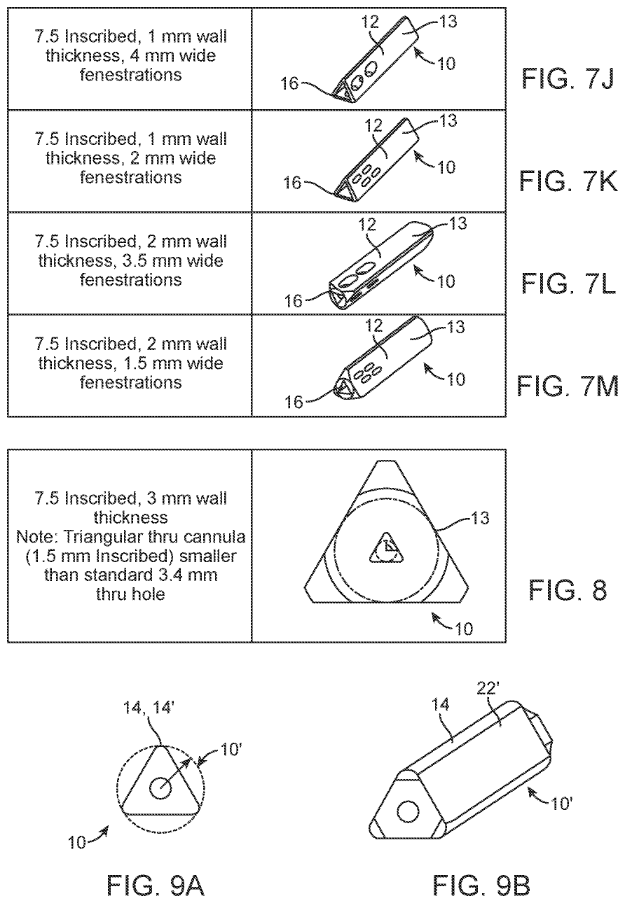

FIGS. 7A and 7B illustrate implants 10 that are similar to the implant 10 illustrated in FIG. 4. The implants 10 in FIGS. 7A and 7B have an elongate body 12 and a triangular cross-sectional area transverse to a longitudinal axis that extends through the elongate body 12. The corners or apices 14 of the implant 10 can be rounded and the distal ends 16 of the implants can be tapered to facilitate insertion. The implants 10 can have a cylindrical lumen 18 that extends the length of the elongate body 12. The difference between the implants 10 in FIGS. 7A and 7B is the size of the implants, in particular with respect to the implant diameter, which can be described with reference to an inscribed circle within the cross-sectional area of the implant. The implant 10 in FIG. 7A has a 7.0 mm inscribed diameter, while the implant in FIG. 7B has a 7.5 mm inscribed diameter. As illustrated, the length of both the implants is 55 mm. It should be noted that the dimensions of the implants described herein can vary. For example, the inscribed diameter can vary between about 3 mm to 14 mm, and the length can vary between about 20 mm to 90 mm, or between about 20 mm to 220 mm.

FIGS. 7C-7F illustrate implants 10 with fenestrations 20. FIG. 7C illustrates an implant 10 that is similar to the implant described in FIG. 7B except that this implant also has elliptical fenestrations 20 located in the distal portion of the implant. Distal fenestrations will preferentially allow for bony through-growth within the sacral portion of the implant. The elliptical fenestrations 20 can be positioned in the center of each face 22 of the elongate body 12 such that the major axis of the ellipse is aligned with the longitudinal axis of the implant. Each face 22 can have between 1 to 4 fenestrations. As illustrated, each face 22 has two fenestrations 20 which are 30 degree ellipses. The ellipse can have a degree between about 5 and 90 degrees, where the degree specifies the angle with which a circle is viewed, meaning a 90 degree ellipse specifies a circle while a zero degree ellipse specifies a line. In FIG. 7D, the fenestrations 20 are still centered on each face but are moved all the way to the distal end 16 of the implant such that the fenestrations 20 extend proximally along the face 22 from the distal end 16. In FIG. 7E, the fenestrations 20 are located at the distal end 16 of the implant but are located at the apices 14 of the implant rather than the center of each face 22. In FIG. 7F, the fenestrations 20 are circular.

The shape, size, number and location of the fenestrations can affect the strength of the implant and its ability to resist or tolerate compressive, tensile, bending, torsional, and shear forces. Other fenestration shapes include rectilinear shapes, such as square, rectangular, triangular, and the like. Other locations for the fenestrations include the middle and proximal portions of the implant. Other fenestration locations will allow for bony through-growth in those portions of the implant, such as the ilium portion of the implant. The depth of the fenestrations can penetrate to the lumen or can stop before reaching the lumen. In some embodiments, the size of the fenestrations facilitates the use of bone graft material or other biologic aids. For example, the fenestrations can be between about 1-10 mm in length and about 1-5 mm in width.

FIGS. 7G and 7H illustrate curved implants 10, where the elongate body 12 has a curvature along its length from the proximal end of the implant to the distal end of the implant. The arc can range from 20 mm to 90 mm, and the radius of curvature can range from about 25 mm to about 1000 mm. The arc can be between about 5 to 45 degrees, or about 15 to 30 degrees. The curved implants 10 can have a cross-sectional area that includes one or more apices. For example, as illustrated, the overall cross-sectional area is triangular. In some embodiments, the curved implant 10 can have an overall cross-sectional area that is circular, such that the implant can be a curved rod. The curved implant 10 can have a curved lumen 18 with a curvature that matches the curvature of the implant, as illustrated in FIG. 7G. Alternatively, the curved implant 10 can have a straight lumen 18, as illustrated in FIG. 7H. In other embodiments, the curved implant 10 can have a curved lumen 18 with a curvature that is different from the curvature of the implant 10. The curvature of the implant can improve resistance to twisting forces, rotation, and pull out forces as compared to a straight rod. The method of implantation of the curved implants 10 may be substantially different from the implantation of straight implants, as further described below. Curved implants have also been described in co-pending U.S. patent application Ser. No. 14/216,938, which is herein incorporated by reference in its entirety for all purposes.

FIG. 7I illustrates an implant 10 that can have a tapered elongate body 12. The elongate body 12 can be tapered from the proximal end 15 to the distal end 16 of the implant. In some embodiments, the taper can be constant such that the faces 22 of the implant are flat. In other embodiments, the taper can result in concave or convex faces 22. For example, in some embodiments, the proximal end 15 can inscribe an 8.5 mm diameter circle, while the distal end can inscribe a 7.5 mm circle. In some embodiments, the difference in diameters between the proximal end and the distal end can be between about 0.5 mm to about 3 mm, or between about 1-2 mm. The taper can facilitate the initial insertion of the implant 10 into a bore, while also providing enhanced securement and friction fit or press-fit within the bore as the wider portion of the implant is driven into the bore.

FIGS. 7J-7M illustrate implants 10 with a hollow interior in place of the lumen such that the elongate body 12 is formed by a plurality of walls. The walls can have a thickness between about 0.5 mm to about 5 mm, or between about 1 mm to about 3 mm. For example, the implants 10 illustrated in FIGS. 7J and 7K have a wall thickness of 1 mm while the implants 10 illustrated in FIGS. 7L and 7M have a wall thickness of 2 mm. The hollow interior of the implant 10 can be filled and packed with bone graft material and/or another biologic aid to promote bone growth into and throughout the implant 10. The biologic aid can contain bone growth promoters such as bone morphogenetic proteins (BMPs) and/or anti-inflammatory agents, analgesic agents, antibiotics, and anti-microbial agents. In addition, bone graft material and/or a biologic aid can be introduced into the bone cavity and/or the joint or space between the two bones through the hollow interior and fenestrations 20. The fenestrations can be between 1 to 10 mm in length. For example, as illustrated, the fenestrations 20 can be 1.5, 2, 3.5 or 4 mm in length. In some embodiments, the distal end 16 of the walls 13 can be sharpened into chisel or blade-like edges such that the implant can be driven into and through the bone at the implantation site without the need of a pre-formed bore. In some embodiments, a pilot bore can be drilled and the implant can be hammered or tapped into the pilot bore without the need of shaping the pilot bore with a broach. The thickness of the walls 13 can be designed to provide enough strength to accommodate the forces exerted on the implant after implantation. FIG. 8 illustrates a cross-sectional view of an implant 10 with 3 mm thick walls, which provides additional strength to the implant 10. A round guide pin can be used to guide placement of the implant 10, or alternatively, a triangular (or rectilinear or another geometry) pin can be used to guide implant placement. Using a triangular or rectilinear guide pin can allow the operator to align the rotational placement of the implant by simply aligning the guide pin. Implants with fenestrations and a hollow interior defined by a plurality of walls have also been described in U.S. Publication No. 2013/0296953, which is herein incorporated by reference in its entirety.

FIGS. 9A and 9B illustrate a series of implants 10, 10′ where the tips of the apices 14, 14′ can be aligned. The smaller of the two implants 10 can be a standard triangular or rectilinear implant as illustrated in solid lines in FIG. 9A, while the larger implant 10′ is illustrated in dotted lines in the same figure. As illustrated, the apices 14, 14′ of the two implants 10, 10′ are located in the same location, but the apices 14′ of the larger implant 10′ are more rounded or blunt and the faces 22′ are spaced further apart such that the larger implant 10′ inscribes a larger circle than the smaller implant 10 even though the apices 14, 14′ are located at the same location. In other embodiments, there can be some variation between the location of the apices while the other features are still retained, such as the rounder apices and larger inscribed circle. This geometry can reduce implant to implant interference when implanting a plurality of implants across the limited space of a single joint. In addition, this geometry may be particularly useful for the rescue of a failed implant where one implant needs to be replaced without interfering with the other neighboring implants. The replacement implant should be larger in size to ensure a tight fit into the replacement bore while also not extending too far as to interfere with neighboring implants.

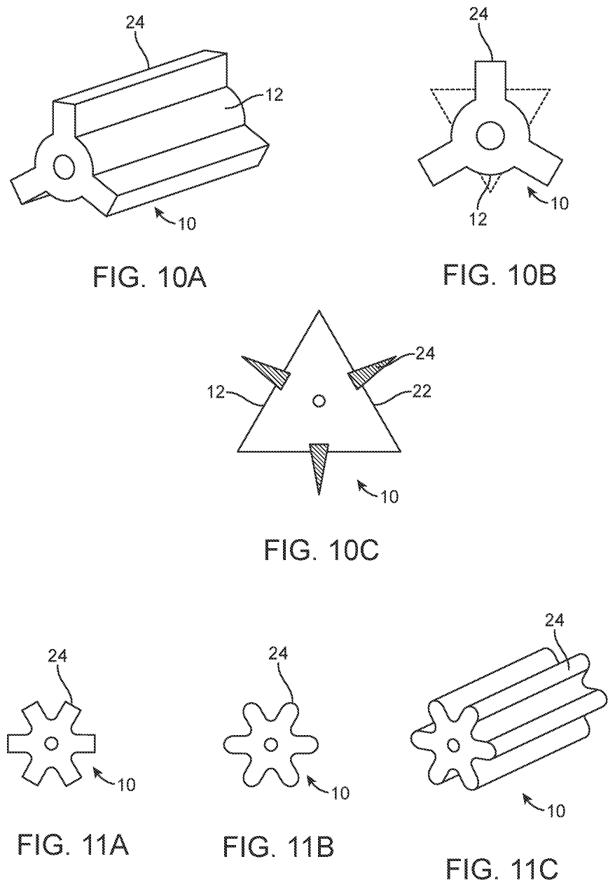

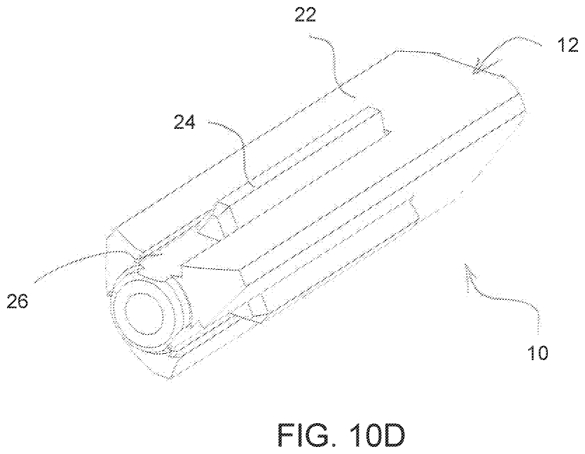

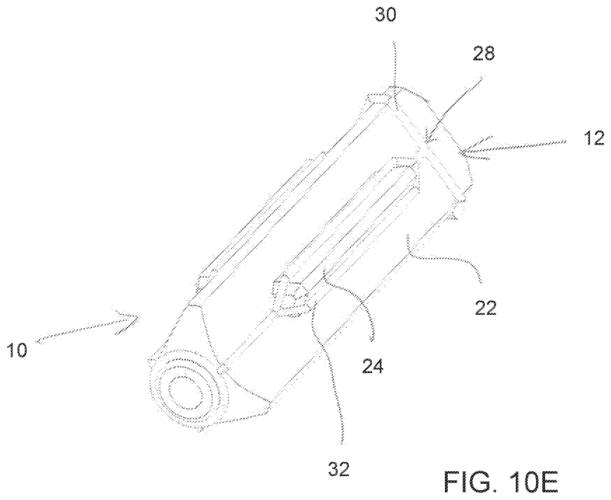

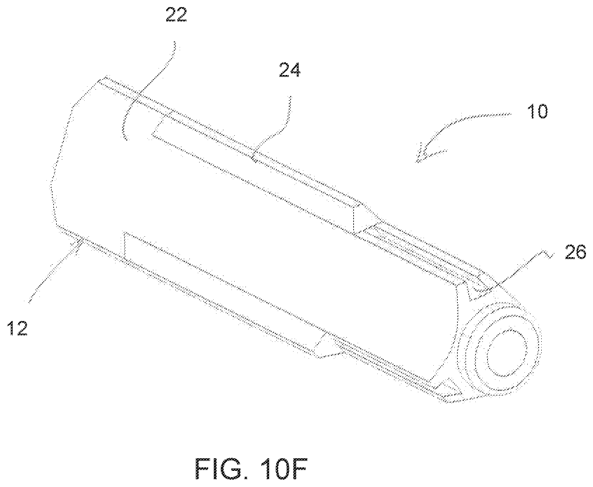

Other implants that may be particularly suitable for the rescue of failed implants or can be used as new implants are shown in FIGS. 10A-13B. For example, FIGS. 10A and 10B illustrate a cylindrical implant 10 with a plurality of ribs 24 that extend from the elongate body 12 of the implant. As illustrated, the implant 10 can have a number of ribs 24 that equals the number of sides of the cavity or the implant that is being rescued or replaced. For example, a three rib 24 implant 10 can be used to rescue or replace a triangular implant or a cylindrical implant. The cylinder portion of the implant 10 can have a diameter that roughly matches the size of the inscribed circle of the triangular implant or the size of the cylindrical implant. In this arrangement, the ribs 24 can be located in bone while the cylindrical portion can be located in the cavity after the old implant is removed. In some embodiments, the ribs 24 can be about 1-5 mm wide and 2-8 mm high. The ribs 24 can have a rectilinear overall cross-sectional profile or can have a rounded profile or can have a profile that is a combination of the two.

FIG. 10C illustrates another embodiment of an implant 10 with a plurality of ribs 24. In this embodiment, the implant 10 has an elongate body 12 with a rectilinear overall cross-section profile, which can be triangular as shown, or can be rectangular or square or another shape. Each face 12 of the elongate body 12 can have a rib 24 which can be located along the center or central portion of the face 12. The ribs 24 can extend the full length of the elongate body or can extend only partially along the length of the elongate body. In some embodiments, the ribs 24 can extend from the proximal ends of the elongate body 12. In other embodiments, the ribs 24 can extend from the distal ends of the elongate body 12. In some embodiments, the ribs 24 can be located along a central portion of the elongate body 12 and be offset from both ends of the implant. The ribs 24 can be tapered to an edge such that the ribs are wedge shaped. The ribs 24 can be attached to the elongate body 12 in a variety of ways. For example, if the ribs 24 are made of a different material than the elongate body, such as being made from a bone graft material including cortical or dense cancellous bone, the ribs 24 can be attached using a dovetail joint or a t-slot fastener. If the ribs 24 are made of the same material as the elongate body 24, the ribs 24 can be attached as described above, or can be fabricated along with the body such that the ribs 24 are integral with the elongate body. The ribs can be made of a variety of materials including bone, metal, metal alloy, ceramic, hydroxyapatite and polymer. The ribs 24 can be designed to cross the joint or fracture during or after implantation.

FIG. 10D illustrates another embodiment of an implant with ribs similar to the embodiment shown in FIG. 10C. The implant 10 has dovetail grooves 26 that run along a length of the elongate body 12 of the implant 10. In this embodiment, the grooves 26 are located on and centered on the face 22 of the elongate body, extending from the distal end of the implant and extending towards the proximal end of the implant. In this embodiment, the grooves 26 terminate before reaching the proximal end of the implant so that when the ribs 24 are fully inserted into the dovetail groove, the ribs 24 are located along a central portion of the elongate body 12 such that the ribs 24 are offset from both ends of the implant. The ribs 24 are designed to fit within the dovetail groove and extend outwards in a wedge shape. In this embodiment, the ribs 24 can terminate in a flat or rounded end instead of a point. The distal ends of both the implant and the ribs can be tapered to narrow in the distal direction to facilitate insertion of the implant. In some embodiments, the width of the base of the rib that fits into the groove can be about ⅛ to ¾, or about ¼ to ½ the width of the face of the implant.

FIG. 10E illustrates another embodiment of an implant with ribs. The ribs 24 can be located along the faces 22 of the implant 10, and can have a similar shape and configuration as the ribs described in FIG. 10D. However, instead of using a dovetail groove to secure the ribs 24, the implant 10 has a frame structure 28 for securing the ribs 24 to the faces 22 of the implant. The frame structure 28 can have an implant securing portion 30 that can be attached to the implant. For example, the implant securing portion 30 can be sized and shaped to receive the implant, or in other words, it can have an opening that matches the cross-sectional profile of the implant. The implant securing portion 30 can also have one or more rib securing portions 32 for securing the ribs 24 against the faces 22 of the implant. The rib securing portion 32 can have an opening sized and shaped to receive a portion of the rib 24. For an elongate, wedge shaped rib, the opening of the rib securing portion can have a length at least equal to the length of the rib while having a width that is less than the width of the base of the rib. Such a configuration allows the rib to extend partially through the opening of the rib securing portion. In some embodiments, the rib securing portions 32 can be biased towards the faces 22 of the implant such that when the ribs are secured within the rib securing portions, the ribs are pressed against the faces of the implant. The frame structure 28 can be configured to center the ribs on the faces of the implant. In some embodiments, the frame structure 28 can be made of a wire or ribbon cage structure that can slide over the implant to secure one or more ribs to the faces of the implant. The frame can be made of a metal, plastic, or composite material. In some embodiments, the frame can be secured to the implant with an adhesive, by welding it to the implant, or by fitting the frame in grooves or slots in the implant. Similarly, the ribs can also be secured to the implant and/or frame using adhesives, by welding, or by fitting it into a groove or slot.

FIG. 10F illustrates another embodiment of an implant with ribs, this time with the ribs located along the apices of the implant. In this embodiment in particular, the ribs 24 are generally made of a different material than the implant. For example, the ribs can be made of a bone graft material, such as autograft or allograft bone, metal, metal alloy, hydroxyapatite, a ceramic, a polymer, or some other material or a combination of materials. However, in other embodiments, the ribs can be made of the same or similar material as the implant. In this embodiment, a portion of each apex can be removed to allow the ribs to form the apices of the implant. For example, the distal and central portion of the apices can be removed, leaving a proximal portion of the apices to remain. In other embodiments, the entire apex can be removed. In other embodiments, the proximal and central portion of the apices can be removed, leaving a distal portion of the apices to remain. A dovetail groove 26 can extend along the removed portion of the apex. The ribs 24 can have a trapezoidal joint portion extending along the length of the ribs that fits within the dovetail groove 26 in a sliding dovetail joint. The portion of the rib 24 extending outside the joint can by sized and shaped to replace a missing portion of the apex. For example, the outside portion of the rib 24 can be triangular or trapezoidal shaped with a rounded off end and with sides that are flush with the faces 22 of the implant. The distal end of the rib 24 can be tapered or beveled.

FIGS. 11A-11C illustrated alternative embodiments of implants 10 with ribs 24. As illustrated, the implants 10 can have a plurality of ribs 24, or even a single rib. The ribs 24 can have a rectilinear overall cross-sectional profile or a curvilinear overall cross-sectional profile or a combination of the two.

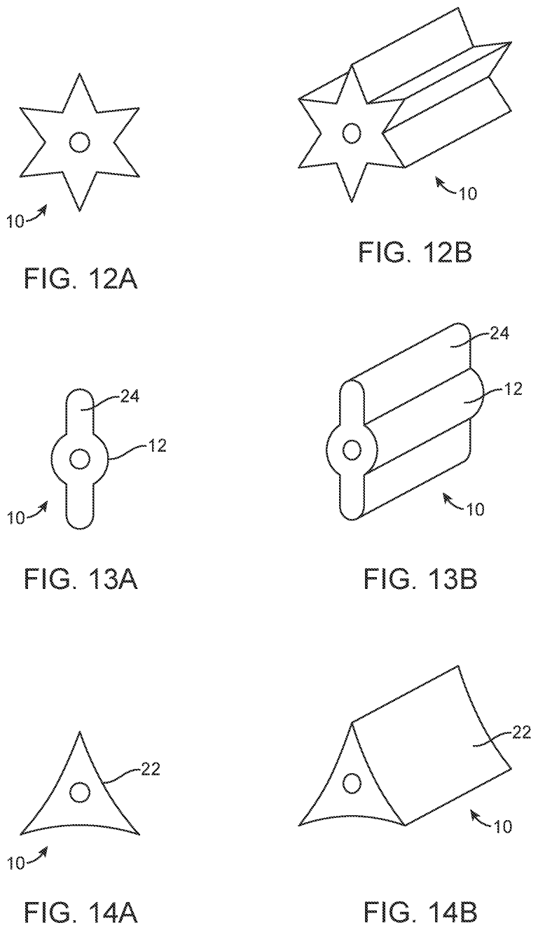

FIGS. 12A and 12B illustrate an embodiment of an implant with a double triangle overall cross-sectional profile. This implant 10 may be particularly suitable for the rescue of a failed triangular implant. The double triangle implant 10 can be inserted into the cavity such that one of the two triangles is aligned with the cavity while the other triangle penetrates into the bone.

FIGS. 13A and 13B illustrates a cylindrical implant 10 with two large curvilinear ribs 24 that are located on opposite sides of the elongate body 12 of the implant 10. This embodiment can be used to rescue a cylindrical implant or a rectilinear implant as long as the ribs 24 are large enough to extend into bone.

FIGS. 14A and 14B illustrate an implant 10 with a triangular overall cross-sectional profile with curved sides or faces 22. The faces 22 can be concave as shown, or can alternatively be convex. An advantage of a convex design is bony preservation, i.e. less bone is removed to prepare the hole for this implant. An advantage of the concave or convex design is the increased surface area compared to an implant with flat surfaces. In some embodiments, the implant 10 can have additional sides or faces or fewer sides or faces. In some embodiments, some of the sides or faces are convex, some of the sides or faces are concave, and/or some of the sides and faces are flat.

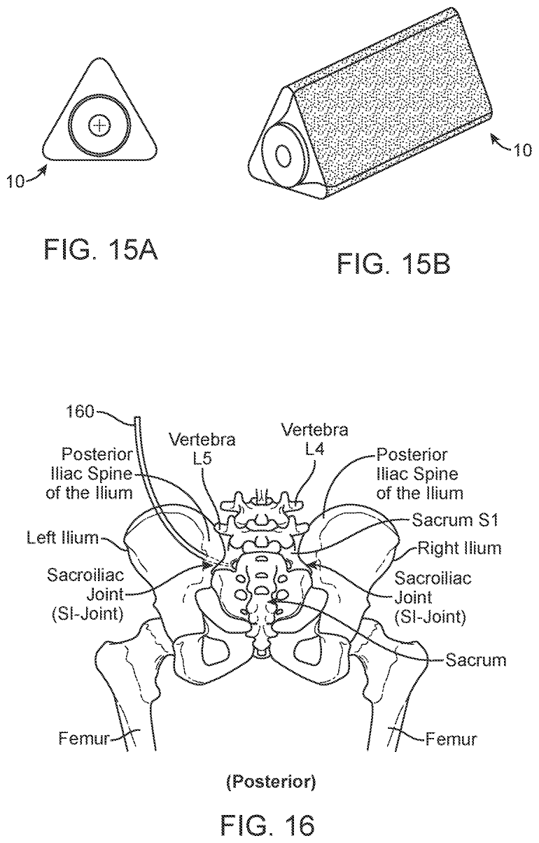

FIGS. 15A and 15B illustrate an implant 10 that is coated with both a titanium plasma spray (TPS) and a hydroxyapatite coating. The TPS coating can provide a roughened and porous surface that facilitates and promotes bony ingrowth and can also provide a roughened surface that can serve as a substrate for the hydroxyapatite coating, which can further promote bony ingrowth and fusion with native bone. The hydroxyapatite coating can be applied over the entire implant, including the exterior surfaces and the interior surfaces, or the hydroxyapatite coating can be applied preferentially on certain surfaces, such as the exterior surface or even just the distal, middle, and/or proximal portions of the implant. The thickness of the TPS coating can be between about 100 and 1500 μm and the thickness of the hydroxyapatite coating can be between about 10 and 1000 inn. These coatings can be applied on any of the implants described herein including implants that are manufactured via 3-D printing or additive manufacturing.

### Methods of Implantation

The methods of implantation of the various implants described herein are described in U.S. Patent Publication No. 2011/0087294, U.S. Pat. Nos. 8,425,570, 8,444,693, 8,414,648, and 8,470,004, and co-pending U.S. Provisional Application No. 61/891,326, each of which is herein incorporated by reference in its entirety for all purposes. These methods are particularly suited for use with straight implants.

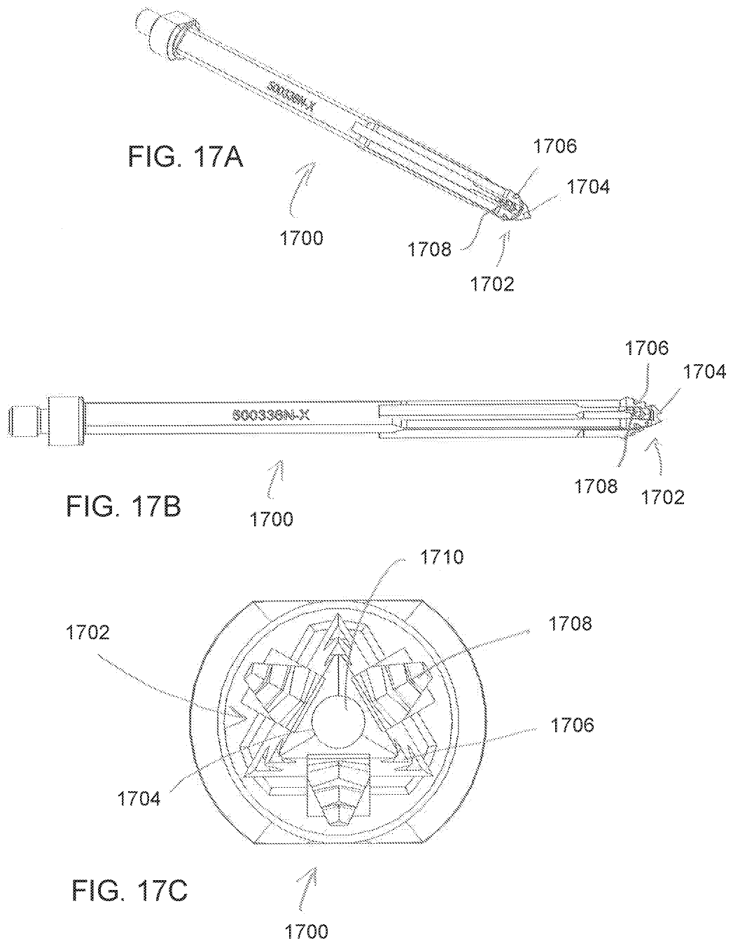

Referring to FIGS. 17A-17C, a modified broach 1700 can be used to cut out and/or shape a bore for an implant with ribs, such as the implants described in FIGS. 10C-10E, for example. The broach can have a distal cutting head portion 1702 with sharp, pointed cutting elements 1704 at the distal end of the cutting head portion. This allows the broach to be used both with a drill to shape a pilot bore and without a drill to form the bore directly over the guide pin. A plurality of cutting elements 1706 are arranged in a progressively expanding outward pattern that matches the cross-sectional shape and size of the implant. In addition, secondary cutting elements 1708, which may be removably or permanently attached to the broach, can be added to cutting head 1702 to cut out slots for the ribs of the implant. The secondary cutting elements 1708 can be positioned between the apices of the cutting elements 1706 to match the position of the ribs on the implant. The secondary cutting elements 1708 can also have a plurality of staggered cutting edges and surfaces that successively increase in size and shape to match the size and shape of the ribs of the implant. The broach 1700 can also have a lumen 1710 extending along the length of the broach for receiving a guide pin. The broach can be used with a modified soft tissue protector that allows the broach to pass through with the secondary cutting elements.

The curved implants illustrated in FIGS. 7G and 7H may require modifications to the method of insertion protocols. Because the implants are curved, it may not be possible or desirable to attempt to hammer or tap the implant into the bone along a straight path using a straight guide pin, a straight drill, a straight broach and the like. Instead, it may be desirable to create and form a curved insertion path that matches the curvature of the implant.

For example, the tooling used to create the curved insertion path can have a radius of curvature that matches the radius of curvature of the implant. For example, some or all of the tooling and the implant can have a matching radius of curvature, as shown in FIG. 16. The tooling, which can include a guide pin or guidewire 160, a tool guide, a drill bit, a broach, and impact hammer and the like can be rotatably secured by an arm with a length equal to the radius of curvature, with one end of the arm attached to a pivot and the other end used to secure the tools and/or implant.

The rotating arm can be used to drive a curved guide pin into the bone to create a curved path through the bone, such as the ilium and the sacrum. A relatively short drill bit with a lumen for receiving the guide pin can be disposed over the curved guide pin to drill out a curved pilot bore. In some embodiments, the drill bit can be secured by the pivoting arm at the end of a curved guide and can be used to drill the curved pilot bore without the insertion of the curved guide pin.

For a curved implant with a circular overall cross section, the curved implant can then be advanced over the curved guide pin and into the curved insertion path that is formed by the curved pilot bore. In some embodiments, the curved implant can be held by the pivoting arm and inserted into the curved insertion path without the aid of a guide pin by rotating the curved arm.

For a rectilinear implant or more broadly a noncircular implant, the curved pilot bore can be shaped using an appropriately shaped broach that matches the cross-sectional shape of the implant. A curved broach, or a short broach, can be advanced over the curved guide pin if present, otherwise the curved broach or short broach can be held in the pivoting arm and advanced through the pilot bore by rotation of the pivoting arm. As the broach is advanced, it shapes the pilot bore into a shape that matches the shape of the implant. In some embodiments, a broach with a sharp distal tip can be used to create a bore without the need of drilling out a pilot bore. Examples of sharp tipped broaches are disclosed in U.S. application Ser. No. 14/216,790, which is herein incorporated by reference in its entirety. The sharp tipped broach can be advanced over the guide pin and can be tapped directly into the bone to form the curved bore.

The curved implant can then be advanced over the curved guide pin and into the curved insertion path that is formed by the curved pilot bore. In some embodiments, the curved implant can be held by the pivoting arm and inserted into the curved insertion path without the aid of a guide pin by rotating the curved arm.

More generally, the implants described herein can be used to fuse any two bone segments, such as two bones that form a joint or two bones resulting from a fracture.

### Implant Rescue

In some cases, an implant will fail to properly fuse with the native bone, which can result in a loose implant and can threaten the stability of the fused bone segments. One solution is to remove the implant and replace it with another implant. The old implant can be removed by chiseling the implant out of bone using a bladed revision tool, such as previously described in co-pending U.S. patent application Ser. No. 14/217,008, which is herein incorporated by reference in its entirety for all purposes.

The implant can then be pulled out of the bone leaving an empty cavity. The cavity is generally of the same shape of the implant, but may be slightly larger due to the bone cutting process. Therefore, the replacement implant that is selected to replace the old implant can be of similar shape but with slightly larger dimensions. For example, an implant that inscribes an 8 mm circle may be suitable as a replacement for an implant that inscribes a 7.5 mm circle. Similarly, a tapered implant such as the tapered implant shown in FIG. 7I can be used. The narrow distal end of the tapered implant can be more easily inserted into the cavity and as the tapered implant is inserted into the cavity, the wider proximal portion can form a secure fit in the cavity.

Alternatively, an implant with a different shape can be inserted into the cavity such that at least one or more portions of the new implant extends into the existing host bone surrounding the cavity. For example, removal of a triangular implant leaves a cavity with a triangular cross-section. Any of the implants illustrated in FIGS. 9A-14B can be inserted into the cavity. For example, any of the implants with ribs can be selected such that the ribs are embedded into the bone surrounding the cavity after insertion into the cavity. In addition, any gaps remaining in the cavity from differences in geometry between the implant and the cavity can be filled with bone graft material and/or a biologic aid. The number of ribs can correspond to the number of sides of the cavity and can be positioned such that each rib is aligned with the center of each side of the cavity.

The implant may be directly tapped into the cavity using a slap hammer or other impact device. In other embodiments, the cavity can be additionally shaped to better receive the implant. For example, a specialized broach with cutouts for the ribs can be used to cut slots into the bone surrounding the cavity, and then the implant can be inserted by aligning the ribs of the implant with the newly cut slots.

The implants can be made of a metal such as titanium, titanium alloy, steel, steel alloy, and the like. Alternatively, the implants can be made of a ceramic, polymer, or bone graft material.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

1-27. (canceled)

28. An implant for bone fixation, the implant comprising:
an elongate body having a longitudinal axis, a distal end, a proximal end, and a cross-section transverse to the longitudinal axis, the transverse cross-section having an inner circular portion and at least three ribs extending radially outward from the inner circular portion,
wherein each of the at least three ribs has a rectilinear overall cross-sectional profile,
each of the at least three ribs being about 1 mm to about 5 mm wide and about 2 mm to about 8 mm high,
the elongate body comprising a plurality of fenestrations located at least near the distal end of the at least three ribs, the plurality of fenestrations sized and configured to allow for bony through-growth, and
the elongate body having a length between about 20 mm and about 90 mm and an inscribed diameter between about 3 mm and about 14 mm.

29. The implant of claim 28, wherein the elongate body is sized and configured to be implanted across a sacroiliac joint.

30. The implant of claim 28, wherein the at least three ribs extend along a full length of the elongate body.

31. The implant of claim 28, wherein the at least three ribs extend only partially along a length of the elongate body.

32. The implant of claim 28, wherein the transverse cross-section has exactly three ribs, and wherein the three ribs are evenly spaced around the inner circular portion.

33. The implant of claim 28, wherein the transverse cross-section has exactly four ribs, and wherein the four ribs are evenly spaced around the inner circular portion.

34. The implant of claim 28, wherein the elongate body includes a central lumen extending from the proximal end to the distal end.

35. The implant of claim 28, wherein the distal end of each of the at least three ribs is tapered.

36. The implant of claim 38, wherein the fenestrations are located on the at least one rib.
